(19) United States
(12) Patent Application Publication
(10) Pub. No.: US 2021/0311043 A1
(43) Pub. Date: Oct. 7, 2021

(54) METHOD FOR DETERMINING A LECTIN-BINDING GLYCAN INDICATIVE TO TRAUMATIC BRAIN INJURY

(71) Applicant: Medicortex Finland Oy, Turku (FI)

(72) Inventors: Adrian HAREL, Turku (FI); Mårten KVIST, Turku (FI); Lasse VÄLIMAA, Turku (FI); Begüm UTZ, Turku (FI); Oskar HAAVISTO, Turku (FI); Venla-Mari NURMI, Turku (FI)

(21) Appl. No.: 16/840,931

(22) Filed: Apr. 6, 2020

(51) Int. Cl. G01N 33/543; G01N 33/53; G01N 33/68; A61B 5/00

(52) U.S. Cl. CPC G01N 33/54386; G01N 33/5308; G01N 2400/10; A61B 5/4064; G01N 2400/02; G01N 33/6896

(57) ABSTRACT

The present invention relates to a method for determining a lectin-binding glycan indicative to traumatic brain injury (TBI) wherein the determining is based on detection of the lectin-binding glycan in a fluid sample using lectins of similar glycan binding property for tracing and capturing.

### FIELD

The present invention relates to a method for determining a lectin-binding glycan indicative to traumatic brain injury (TBI), in particular to methods wherein the determining is based on detection of the lectin-binding glycan in a fluid sample using lectins of similar glycan binding property for tracing and capturing.

### BACKGROUND

Traumatic brain injury (TBI) is the leading cause of central nervous system impairment in these days, with more than 2.5 million individuals suffering annually from TBI in the US alone. According to the CDC, the highest incidence of TBI occurs among children 0-4 years old, adolescents 15-19 years old, and adults over 65 years of age. Despite the broad range of the population affected, TBI is still under-served and remains an unexplored pathological condition.

Traditionally, TBI has been acutely diagnosed and classified by neurological examinations, such as Glasgow Coma Scale (GCS). However, the use of the GCS as a diagnostic tool is subject to a number of limitations. Recent research has provided evidence that the use of sedative drugs precludes accurate GCS assessment during the first 24 hours. Further challenges to diagnosis are presented by the evolving nature of some brain lesions, which can lead to further neurological impairment. In addition, neurological responses after TBI can vary over time for reasons unrelated to the injury. Still further challenges include the trauma subject's possible unconsciousness or inability to communicate.

Neuroimaging techniques, such as x-ray, CT scanning and MRI, are used to provide information on injury magnitude and location and are not influenced by the aforementioned disadvantages. However, CT scanning has low sensitivity to diffuse brain damage, and availability and utility of MRI is limited. MRI is also very impractical to perform if subjects are physiologically unstable and can lead to inaccurate diagnoses in military injuries in which metal fragments in the body are common.

Mild and moderate TBI represent more than 90% of TBI injuries; this injury range represents the greatest challenges to accurate acute diagnosis and outcome prediction. Unlike severe TBI, there is no universally recognized neurologic assessment scale such as the GCS, and many cases of mild TBI are classified as subclinical brain injury (SCI). The widespread recognition of inadequate approaches to diagnose mild TBI indicate the need for significant improvement in the diagnosis and classification of TBI, such as the use of biomarkers to supplement functional and imaging-based assessments. These biomarkers can be altered gene expression, protein, lipid or glycan metabolites, or a combination of these changes after a traumatic brain injury, reflecting the initial insult (the primary injury) and the evolution of a cascade of secondary damage (the secondary injury). In particular, subclinical brain injury status or SCI could be diagnosed with a biomarker analysis.

As with many injuries, increased serum levels of cytokines and chemokines have been noted post-TBI and, as such, have been proposed as potential surrogate markers for TBI outcome. However, to date, there are not too many biomarkers for the diagnosis or prognosis of TBI. This is because of several obstacles to the development of reliable blood biomarkers of TBI. For instance, the blood-brain barrier (BBB) hinders the assessment of biochemical changes in the brain by use of blood biomarkers in mild TBI, although impaired BBB integrity, as seen in severe TBI, can increase the levels of brain-derived proteins in the blood. Nevertheless, owing to their dilution in the much larger plasma volume, biomarkers that are highly expressed within the central nervous system exist at very low concentrations in blood. Moreover, some potential biomarkers undergo proteolytic degradation in the blood, and their levels might be affected by clearance from blood via the liver or kidney. As a consequence, reliable blood biomarkers have been extremely difficult to identify.

WO/2016/166419 by the present assignee and one of the present inventors, which is incorporated by reference in its entirety, discloses glycan-based biomarkers for the diagnosis and prognosis of brain damage, such as traumatic brain injury (TBI), subclinical brain injury (SCI) and acquired brain injury (ABI). The glycan-based biomarker protocol disclosed therein may be used as an end point in clinical trials and in other diagnostic tests to determine, qualify, and/or assess brain injury status, for example, to diagnose brain injury, in an individual, subject or patient. As part of the diagnosis afforded by the glycan-based biomarker disclosed therein, brain injury status can include determination of a subject's subclinical brain injury status or SCI status, for example, to diagnose SCI, in an individual, subject or patient.

WO2018154401 by the present assignee and the present inventors incorporated by reference in its entirety, discloses a device for conducting a non-invasive analysis of a bodily fluid, such as saliva or urine, to determine the presence and the level of a certain glycan-based biomarkers that are indicative of brain injury, that are carried by the bodily fluid. The device includes an indicator formulation capable of changing color in response to exposure to the biomarkers to provide a visual indication of the presence and the level of the biomarkers carried by the bodily fluid. The device comprises a porous matrix substrate for establishing a high void volume within the carrier substrate, and an indicator formulation carried by the carrier substrate. The indicator formulation includes a chromogen agent (a visually detectable label) and a biomarker-specific agent selected from a variety of agents responsive to levels of any one of a plurality of different glycan-based biomarkers that are indicative of brain injury.

Historic obstacles to point-of-care devices include manufacturing challenges, ease-of-use limitations, and government regulations. Some of these obstacles have been reduced through advances in technology and recognition by governments and other regulatory bodies of the importance of point-of-care testing. However, important considerations, including ease-of-use and accuracy, still render point-of-care tests unsuitable for many healthcare facilities and domestic settings, and more so for particular medical conditions, such as brain injury.

One challenge is that the biomarkers indicative to TBI are often present also in body fluid of subjects not suffering from TBI. Thus, the presence of the marker in body fluid may not be an indication of TBI. Accordingly, there is still need for further methods for determining brain injury.

### SUMMARY

The present invention is based on the observation that when similar lectins are used for tracing and capturing a lectin-binding glycan indicative to traumatic brain injury, some of the prior art problems can be avoided or at least alleviated.

Accordingly, it is an object of the present invention to provide a method for determining a lectin-binding glycan indicative to traumatic brain injury (TBI) in a fluid sample the method comprising
a) providing
   i. a probe comprising
      a porous matrix, the porous matrix comprising a first part, a second part and a detection zone therebetween and
      a first lectin immobilized to the detection zone, the first lectin adapted to bind to the lectin-binding glycan,
   ii. a conjugate comprising
      a second lectin adapted to bind to the lectin-binding glycan and
      a signal generation means adapted to produce a detectable signal upon binding of the second lectin to the lectin-binding glycan immobilized to the detection zone via the first lectin,
   wherein the first lectin and the second lectin have similar glycan-binding properties,
b) exposing the detection zone to the fluid sample,
c) exposing the detection zone to the conjugate,
d) detecting signal derived from the signal generation means on the detection zone, and
e) determining level of the lectin-binding glycan in the fluid sample based on the detecting.

It is also an object of the present invention to provide a method for determining traumatic brain injury (TBI) in a subject, the method comprising
a) providing
   i. a probe comprising a porous matrix, the porous matrix comprising
      a first part, a second part and a detection zone therebetween and
      a first lectin immobilized to the detection zone, the first lectin adapted to bind to a lectin-binding glycan indicative to traumatic brain injury and
   ii. a conjugate comprising
      a second lectin adapted to bind to the lectin-binding glycan indicative to traumatic brain injury and
      a signal generation means adapted to produce a detectable signal upon binding of the second lectin to the lectin-binding glycan immobilized to the detection zone via the first lectin,
   wherein the first lectin and the second lectin have similar glycan-binding properties,
b) exposing the detection zone to a body fluid sample obtained from the subject,
c) exposing the detection zone to the conjugate,
d) detecting signal derived from the signal generation means at the detection zone,
e) determining level of the lectin-binding glycan in the body fluid sample based on the detecting,
f) comparing the level of the lectin-binding glycan in the sample to a control level indicative to a level of the lectin-binding glycan in body fluid of a subject not suffering from TBI and
h) determining TBI in the subject based on the comparing, wherein elevated level is an indication of TBI in the subject.

Further objects of the present invention are described in the accompanying dependent claims.

Exemplifying and non-limiting embodiments of the invention, both as to constructions and to methods of operation, together with additional objects and advantages thereof, are best understood from the following description of specific exemplifying embodiments when read in connection with the accompanying drawings.

The verbs “to comprise” and “to include” are used in this document as open limitations that neither exclude nor require the existence of un-recited features. The features recited in the accompanied depending claims are mutually freely combinable unless otherwise explicitly stated. Furthermore, it is to be understood that the use of “a” or “an”, i.e. a singular form, throughout this document does not exclude a plurality.

### BRIEF DESCRIPTION OF THE DRAWINGS

FIG. 1 illustrates a workflow of a lectin sandwich assay protocol according to an exemplary non-limiting embodiment of the present invention,

FIG. 2 illustrates an exemplary test strip for determining increased level or normal level of a lectin-binding glycan in a fluid sample,

FIG. 3 illustrates a flowchart of a method according to an exemplary non-limiting embodiment of the present invention for determining the presence or absence of TBI in a subject,

FIG. 4 illustrates an exemplary test strip for determining the presence or absence of traumatic brain injury in a subject, and

FIG. 5 illustrates results from lectin capture-tracer pair comparison quantitated with ImageJ software (TBI=sample from a subject suffering from traumatic brain injury; HC=healthy control sample from a subject not suffering from traumatic brain injury).

### DESCRIPTION

According to one embodiment, the present invention concerns a method for determining a lectin-binding glycan indicative to traumatic brain injury (TBI) in a fluid sample. The method comprises the following steps:
a) providing
   i. a probe comprising a porous matrix comprising
      a first part, a second part and a detection zone therebetween and
      a first lectin immobilized to the detection zone, and
   ii. a conjugate comprising
      a second lectin and
      a signal generation means adapted to produce a detectable signal upon binding of the second lectin to the lectin-binding glycan to the detection zone via the first lectin
   wherein the first lectin and the second lectin have similar glycan-binding properties,
b) exposing the detection zone to the fluid sample,
c) exposing the detection zone to the conjugate,
d) detecting signal derived from the signal generation means at the detection zone, and
e) determining level of the lectin-binding glycan in the fluid sample based on the detecting.

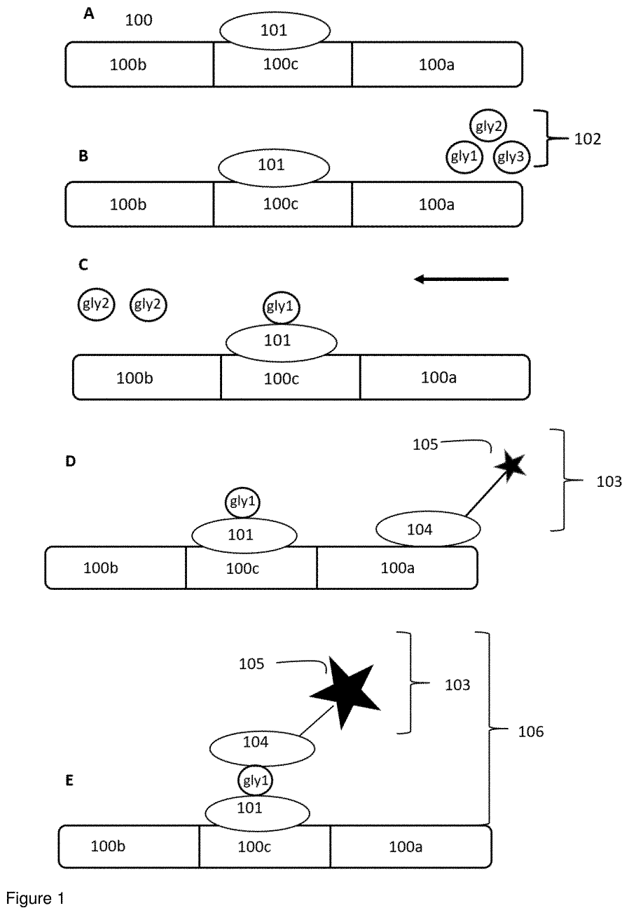

An exemplary non-limiting workflow of the method is shown in FIG. 1. The arrow in the figure shows the flow direction.

FIG. 1A shows a probe comprising a porous matrix 100. The porous matrix comprises a first part 100a, a second part 100b and a detection zone 100c therebetween. A first lectin 101 is immobilized on the detection zone. The first lectin is adapted to bind the lectin-binding glycan indicative to TBI. The binding is selective.

FIG. 1B shows a situation where a fluid sample 102 comprising three lectin-binding glycans (gly1, gly2, gly3) has been applied on the first part of the porous matrix. The sample is allowed to elute from the first part of the matrix towards the second part through the detection zone. The aim is to expose the detection zone comprising the immobilized first lectin to glycans of the fluid sample.

FIG. 1C shows a situation where one of the glycans (gly1) has bound to the immobilized lectin 101. Other glycans do not bind to the immobilized lectin but elute through the detection zone to the second part. Removal of the unbound glycans and other material present in the sample can be performed by e.g. by applying a wash solution to the first part of the porous matrix and allowing the wash solution to elute through the detection zone towards the second part, i.e. to flush the matrix. According to another exemplary embodiment the removal of the unbound glycans and other material is performed by soaking the probe to a wash solution. An exemplary was solution is saline such as PBS.

FIG. 1D shows a situation where the unbound glycans (gly2 and gly3) have been washed away, and a conjugate 103, or a solution comprising the conjugate comprising a second lectin 104 and a signal generation means 105 has been applied on the first part of the porous matrix. The second lectin is adapted to bind the lectin-binding glycan. The binding is selective.

The immobilized lectin, i.e. the first lectin and the second lectin have similar glycan-binding properties. According to a particular embodiment the lectins are similar lectins. The conjugate is adapted to elute from the first part towards the second part through the detection zone.

FIG. 1E shows a situation where the conjugate has eluted from the first part to the detection zone. The second lectin 104 of the conjugate binds to the glycan (gly1) immobilized to the detection zone via the first lectin 101. Removal of the unbound glycans and other material present in the sample can be performed by e.g. by applying a wash solution to the first part of the porous matrix and allowing the wash solution to elute through the detection zone towards the second part, i.e. to flush the matrix. The binding causes increase of concentration of signal generating means at the detection zone. The level of the signal is dependent on the amount of the lectin-binding glycan (gly1) in the sample.

### The Fluid Sample

The fluid sample can be any sample expected to include glycans, in particular lectin-binding glycans indicative to TBI. Particular lectin-binding glycans comprise sequences listed in Table 1.

A particular fluid is body fluid. Exemplary body fluids are saliva, urine, serum, blood plasma, tears, cerebrospinal fluid, oral secretions, respiratory secretions. A particular body fluid is saliva. Another particular body fluid is urine.

According to one embodiment the method comprises exposing the fluid sample to N-acetyl-L-cysteine before exposing the detection zone. This may be useful if the fluid sample, such as saliva or sputum, is viscous. N-acetyl-L-cysteine is able to disintegrate mucous proteins.

### The Porous Matrix

The porous matrix can be made from any material capable of absorbing liquid. The porosity of the material can be unidirectional (i.e., with pores or fibers running wholly or predominantly parallel to an axis of the member) or multidirectional (omnidirectional, so that the member has an amorphous sponge-like structure). Porous plastics material, such as polypropylene, polyethylene (preferably of very high molecular weight), polyvinylidene fluoride, ethylene vinyl acetate, acrylonitrile and polytetrafluoroethylene can be used. It can be advantageous to pre-treat the porous matrix with a surface-active agent during manufacture, as this can reduce any inherent hydrophobicity in the member and therefore enhance its ability to take up and deliver a moist sample rapidly and efficiently. The porous matrix can also be made from paper or other cellulosic materials, such as nitrocellulose. Materials that are widely used in the nibs of so-called fiber tipped pens are particularly suitable since such materials can be shaped or extruded in a variety of lengths and shapes. Preferably the porous matrix should be chosen such that the porous material of the matrix can be saturated with aqueous liquid within a matter of seconds. Preferably the material remains robust when moist.

In some embodiments, the porous matrix is selected from the family of nitrocellulose materials. This family has some advantage over conventional synthetic or cellulose materials, such as paper, because it has a natural ability to bind proteins without requiring prior sensitization. The two or more lectins can be applied directly to nitrocellulose and immobilized thereon. No chemical treatment is required which might interfere with the essential specific binding activity of the two or more lectins. Unused binding sites on the nitrocellulose can thereafter be blocked using simple materials, such as polyvinyl alcohol. Moreover, nitrocellulose is generally safe, non-toxic and readily available in a range of pore sizes and this facilitates the selection of a carrier material to suit particularly requirements such as sample flow rate.

Preferably the porous matrix has a pore size of at least one micron. Preferably the porous matrix has a pore size not greater than about 20 microns. In some embodiments, the average pore size of the porous matrix ranges 1-10, 1-20, 1-30, 1-40 or 1-50 microns.

Blocking of unused binding sites in the porous matrix can be achieved by treatment with protein (e.g. bovine serum albumin or milk protein), or with polyvinyl alcohol or ethanolamine, or any combination of these agents, for example. The mobile reagent(s) can then be dispensed onto the dry matrix and will become mobile in the carrier when in the moist state. Between each of these various process steps (sensitization, application of unlabeled reagent, blocking and application of the labeled reagent), the porous matrix should be dried.

### Immobilization of the Lectin

The lectin can be applied to the detection zone in a variety of ways. Various “printing” techniques have previously been proposed for application of liquid reagents to porous matrices, e.g. micro-syringes, pens using metered pumps, direct printing and inkjet printing, and any of these techniques can be used in the present context. To facilitate manufacture, the matrix can be treated with the reagents and then subdivided into smaller portions, e.g., small narrow strips each embodying the required reagent-containing zones, to provide a plurality of identical carrier units. In some embodiments, the lectin is permanently immobilized in a detection zone in/on the porous matrix and is therefore not mobile in the moist state e.g. when the probe is soaked with the liquid sample.

### The Signal Generation Means

The signal generation means is typically a label, preferably a visually detectable label. In some embodiments, the lectin is conjugated to the visually detectable label covalently. In some other embodiments, the lectin is conjugated to the visually detectable label non-covalently. The detectable labels are preferably selected such that upon a binding event of the conjugated lectin to the glycan, the visually detectable label has a color intensity level, which has not been visible prior to the binding event, and thus becomes visible thereby making the binding even visibly distinguishable. A particular signal generating means is colloidal gold. In the case of colloidal gold, the visually detectable signal is a result of increase of concentration of the colloidal gold upon binding to the lectin-binding glycan immobilized on the detection zone. Further labels suitable for lateral flow assays have been disclosed in Badir et al. (Trends. Anal. Chem., 82, 2016, pp. 286-306) incorporated here by reference.

The most commonly used detection moieties in lateral flow immunoassays are gold nanoparticles or latex beads. These particles produce a colored readout which requires no development process for visualization. Fluorescent labels, enzymes, other colloidal metals and magnetic particles can also be employed.

In the context of embodiments of the present invention, the term “visible” refer to a visual signal that can be detected by the naked eye (visible light which a human eye can perceive), without the use of additional machinery or processes. In the context of embodiments of the present invention, a visible signal is a change in a color of a certain object or an area thereon, relative to the color that has been characteristic to the object or area prior to the change. A change can also be assessed in comparison to the background of the object or area, and in comparison, to the surrounding of the object or area.

### The Lectins

According to the method the first lectin and the second lectin have similar glycan binding properties. According to a particular embodiment the first lectin and the second lectin are similar lectins. According to one embodiment the first lectin and the second lectin is selected from Aleuria autantia (AAL), Galanthus nivalis (GNA), Hippeastrum hybrid (HHA), Narcissus pseudonarcissus (NPA), Sambucus nigra I (SNA-I), Ulex europaeus I (UEA-I), Urtica dioica (UDA), Allium savitum (ASA), Pisum sativum lectin (PSA) and Phytohemagglutinin-L (PHA-L). According to another embodiment the first lectin and the second lectin is selected from Urtica dioica (UDA), Allium savitum (ASA), Pisum sativum lectin (PSA) and Phytohemagglutinin-L (PHA-L). According to a preferable embodiment the first lectin and the second lectin is UDA.

Table 1 presents nine lectins which have been found to be important biomarkers. Data represent patients suffering from Traumatic Brain Injury (TBI), the statistically significant increase marked with (+) compares TBI-patients relative to healthy controls in urine and saliva.

TABLE 1

| Lectin | Specificity | Urine | Saliva |
|---|---|---|---|
| AAL | Fucα6GlcNAc | + | |
| ASA | αMan | + | + |
| GNA | αMan | + | + |
| HHA | αMan | + | + |
| NPA | αMan | + | + |
| PSA | αMan, αGlc | + | |
| SNA-I | NANAα(2,6)GalNAc > GalNAc = | + | + |
| | Lac > GalNANAα(2,6)Gal | | |
| UDA | GlcNAc | + | + |
| UEA-I | αFuc | + | |

The method comprises exposing the detection zone comprising immobilized lectin to the fluid sample. According to one embodiment the fluid sample is applied directly to the detection zone. According to another embodiment the fluid sample is applied to a first part of the porous matrix and allowed to elute through the detection zone towards the second part. If the fluid sample comprises the lectin-binding glycan, it binds to the immobilized lectin adapted to bind the lectin-binding glycan.

The method comprises exposing the detection zone to a conjugate comprising a second lectin and a signal generation means. According to an exemplary embodiment a solution comprising the conjugate is applied on the first part of the porous matrix and allowed to elute through the detection zone towards the second part of the porous matrix. According to another embodiment the solution is applied directly on the detection zone.

If the detection zone comprises a lectin-binding glycan bound to the immobilized lectin, the conjugate binds also to the lectin-binding glycan immobilized on the detection zone of the porous matrix via the first lectin.

The method comprises also removing unbound material from the detection zone. According to one embodiment the removing comprises flush the probe, in particular the detection zone with a wash solution which could disturb determining of the lectin-binding ligand. An exemplary wash solution is saline, such as phosphate buffered saline.

The method comprises detecting signal derived from the signal generation means on the detection zone after removal of unbound material. If the detection zone comprises the lectin-binding glycan bound the immobilized lectin, the conjugate binds to the lectin-binding glycan, and the binding is detected based on the signal derived from the signal generating means. When the signal generating means is e.g. colloidal gold, the visible signal is result of increase of colloidal gold concentration on the detection zone.

The method comprises determining level of the lectin-binding glycan in the fluid sample based on the detecting. According to an exemplary embodiment the determining comprises calculating increase of the signal of the signal generation means upon the binding event.

According to a particular embodiment the method comprises also steps of
f) comparing the level of the lectin-binding glycan in the fluid sample to a control level, and
g) determining presence or absence of increased level of the lectin binding glycan in the fluid sample based on the comparing.

According to a particular embodiment the control level is the level of the lectin binding glycan in a body fluid of a subject not suffering from TBI.

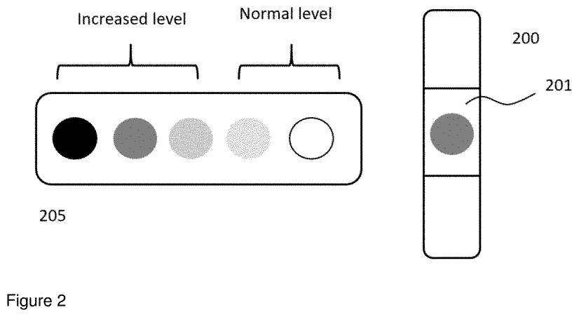

There are different ways to perform steps f) and g). According to one embodiment the steps comprise performing at least two parallel methods: one with a fluid sample with unknown level of lectin-binding glycan indicative to TBI, and another one with body fluid obtainable from a subject not suffering from TBI, or with a solution mimicking a body fluid obtainable from a subject not suffering from TBI. If the level of the lectin-binding glycan is in the fluid sample higher than a predetermined control level e.g. two times higher, the fluid sample has significantly increased level or lectin-binding glycan indicative to TBI. When the signal generating means produces a visually detectable color upon binding to the glycan, the determining can be done by comparing the color obtained to various predetermined colors indicative to different lectin-binding glycan concentrations. The determining can be assisted e.g. with an exemplary test strip 205 as shown in FIG. 2. According to this embodiment, the porous matrix 200 is allowed to dry and the color on the detection zone 201 is compared with the colors of the test strip.

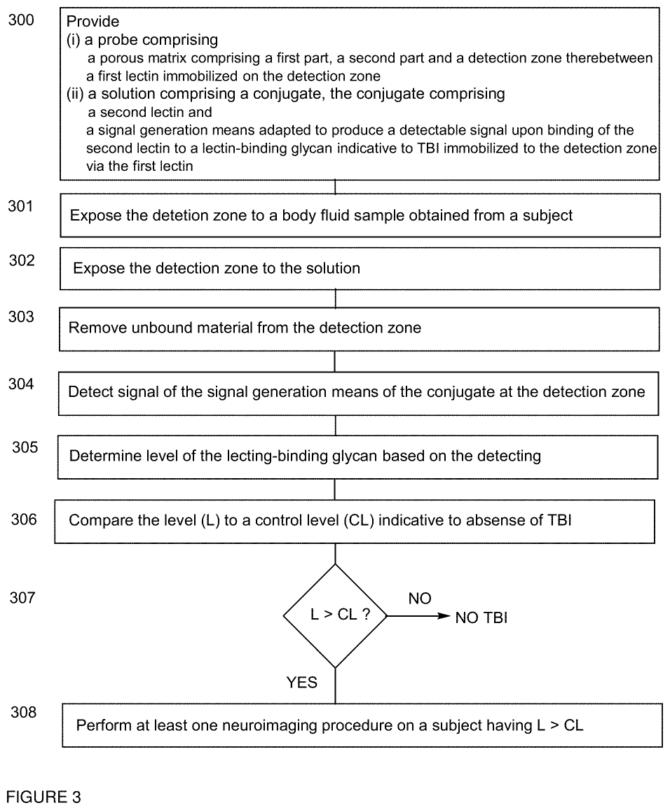

The method of the present invention can also be used for determining traumatic brain injury in a subject. According to this embodiment the steps a)-e) are as disclosed above but steps f) and g) are not optional. The probe, the second lectin and the conjugate may be as disclosed above. The method may also comprise exposing the fluid sample to N-acetyl-L-cysteine before exposing the detection zone. This may be useful if the body fluid sample such as saliva is viscous. The fluid sample is a body fluid sample obtained from a subject suspected to suffer from TBI. An exemplary non-limiting flow chart of the method for determining traumatic brain injury in a subject is shown in FIG. 3. The method comprises the following actions:

Action 300: Provide
i. a probe comprising a porous matrix, the porous matrix comprising
   a first part, a second part and a detection zone therebetween and
   a first lectin immobilized on the detection zone, and
ii. a solution comprising a conjugate comprising
   a second lectin and
   a signal generation means adapted to produce a detectable signal upon binding of the second lectin to a lectin-binding glycan indicative to TBI immobilized to the detection zone via the first lectin,
wherein the first lectin and the second lectin have similar glycan binding properties.

Action 301: Expose the detection zone to a body fluid sample obtained from a subject,

Action 302: Expose the detection zone to the solution.

Action 303: Remove unbound material from the detection zone.

Action 304: Detect signal of the signal generation means of the conjugate at the detection zone.

Action 305: Determine level of the lectin-binding glycan in the sample based on the detecting.

Action 306: Compare the determined level of the lectin-binding glycan in the sample to a predetermined control level indicative to absence of TBI.

Action 307: Determine presence or absence of TBI in the subject based on the comparing.

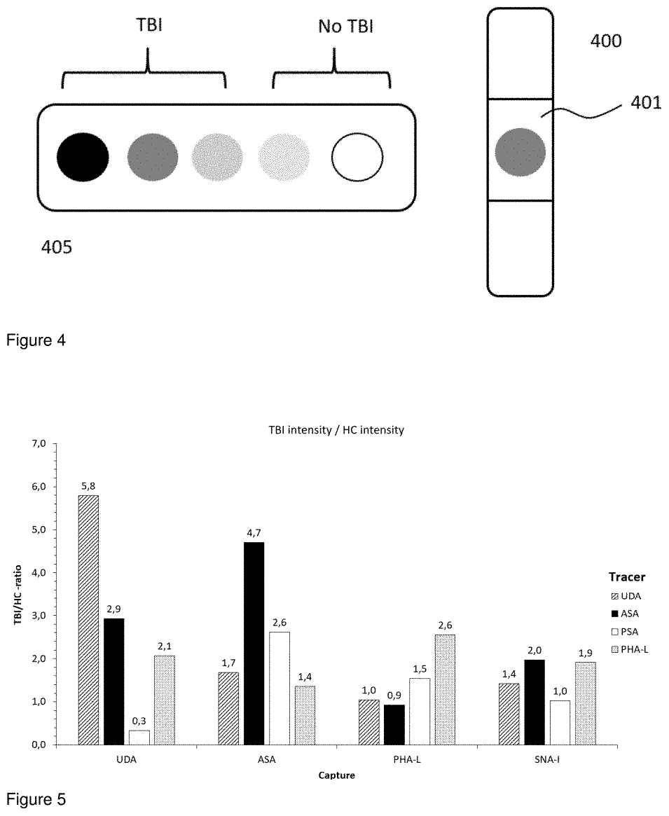

There are different ways to perform Actions 305 and Action 306. According to one embodiment the Actions are done by performing at least two parallel methods: one with a fluid sample with unknown level of lectin-binding glycan indicative to TBI, and another one with body fluid obtainable from a subject not suffering from TBI, or with a solution mimicking a body fluid obtainable from a subject not suffering from TBI. If the level of the lectin-binding glycan in the fluid sample is higher than a predetermined control level e.g. two times higher, the fluid sample has significantly increased level or lectin-binding glycan indicative to TBI. When the signal generating means produces a visually detectable color upon binding to the glycan, the determining can be done by comparing the color obtained to various predetermined colors indicative to different lectin-binding glycan concentrations. The determining can be assisted e.g. with an exemplary test strip 405 shown in FIG. 4. According to this embodiment, the porous matrix 400 is allowed to dry and the color on the detection zone 401 is compared with the colors of the test strip.

If the level of the lectin-binding glycan in the body fluid sample is above a control level, the method preferably includes also subjecting the subject to one or more neuroimaging procedures. According to a particular embodiment the method comprises

Action 308: performing at least one neuroimaging procedure selected from a group consisting of x-ray, computerized tomography, and magnetic resonance imaging on a subject having the elevated level of the lectin-binding glycan.

The neuroimaging procedure is preferably performed if the subject has elevated level of the lectin-binding glycan in the body fluid.

### EXPERIMENTAL

Lectins used were commercially available. Lectin-colloidal gold conjugates were prepared as disclosed in https://www.abbexa.com/lateral-flow-assay

Representative examples of porous matrix materials suitable for the method include paper, nitrocellulose and nylon membranes. Essential features of the material are its ability to bind protein; speed of liquid conduction; and, if necessary, after pre-treatment, its ability to allow the passage of labeled binding reagents therethrough. In embodiments using direct label, it may be desirable for the material to allow flow of particles of size up to a few microns (usually less than 0.5 μm). Examples of flow rates obtained with various materials are presented in Table 2 below, showing the time in minutes to flow through 45 mm of material.

TABLE 2

| | Pore | |
|---|---|---|
| | size | Time |
| Material type | [μm] | [minutes] |
| Whatman ®’s chromatography paper | | |
| (Schleicher & Schuell ®) | 3 | 3.40 |
| unbacked nitrocellulose sheet | 5 | 3.30 |
| | 8 | 3.00 |
| | 12 | 2.20 |
| backed polyester sheet | 8 | 3.40 |
| Whatman ®’s Nitrocellulose sheet | 5 | 19.20 |
| Pall ®’s Immunodyne ® (nylon) | 3 | 4.00 |
| | 5 | 3.20 |

The travel rate of a test procedure will be determined by the flow rate of the material employed and while any of the above materials can be used some will give faster tests than others.

Nitrocellulose is advantageous of requiring no activation and will immobilize proteins strongly by absorption. Immunodyne® is pre-activated and requires no chemical treatment. Papers, such as Whatman® 3MM, require chemical activation with for example carbonyl diimidazole in order to successfully immobilize proteins.

### Example. A Typical Procedure

2-3 μL of 2 mg/mL pure lectin in PBS was pipetted on detection zone of a nitrocellulose matrix and allowed to dry for 30 min at 35° C. Then, 5 μL of sample was pipetted on the lectin. First part of the matrix was soaked to a solution comprising the lectin conjugated with colloidal gold. The solution was allowed to elute from the first part of the matrix towards the second part of the matrix through the detection zone. When the solvent front received second end of the matrix, the matrix was eluted with wash buffer and dried. The procedure was performed using a TBI sample and a HC-sample. The probes were photographed, and visualization was performed using/mageJ-instrument. Results are shown in FIG. 5. As show, the best TBI/HC ratios were obtained when the same lectin was used for tracing and capturing.

The specific examples provided in the description given above should not be construed as limiting the scope and/or the applicability of the appended claims.

1. A method for determining a lectin-binding glycan indicative to traumatic brain injury (TBI) in a fluid sample, the method comprising
a) providing
i. a probe comprising a porous matrix, the porous matrix comprising
a first part, a second part and a detection zone therebetween and
a first lectin adapted to bind the lectin-binding glycan and being immobilized to the detection zone, and
ii. a conjugate comprising
a second lectin adapted to bind the lectin-binding glycan and
a signal generation means adapted to produce a detectable signal upon binding of the second lectin of the conjugate to the lectin-binding glycan immobilized to the detection zone via the first lectin,
wherein the first lectin and the second lectin have similar glycan-binding properties,
b) exposing the detection zone to the fluid sample,
c) exposing the detection zone to the conjugate,
d) detecting signal derived from the signal generation means at the detection zone, and
e) determining level of the lectin-binding glycan in the fluid sample based on the detecting.

2. The method according to claim 1 comprising
f) comparing the level of the lectin-binding glycan in the fluid sample to a control level indicative to a level of the lectin-binding glycan in body fluid of a subject not suffering from TBI and
g) determining presence or absence of increased level of the lectin-binding glycan in the fluid sample based on the comparing.

3. The method according to claim 1 comprising removing unbound material from the detection zone before the detecting of step d).

4. The method according to claim 1 wherein the exposing of step b) comprises applying the fluid sample onto to the detection zone.

5. The method according to claim 1 wherein the exposing of step b) comprises applying the fluid sample onto the first part of the porous matrix and allowing the fluid sample to elute through the detection zone towards the second part.

6. The method according to claim 1 wherein the exposing of step c) comprises applying a solution comprising the conjugate to the first part of the porous matrix and allowing the solution to elute through the detection zone towards the second part.

7. The method according to claim 1 wherein the fluid is body fluid selected from saliva, urine, serum and blood plasma, tears, cerebrospinal fluid, oral secretions, respiratory secretions.

8. The method according to claim 7 wherein the body fluid is saliva.

9. The method according to claim 7 wherein the body fluid is urine.

10. The method according to claim 8 wherein the method comprises exposing the fluid sample to N-acetyl-L-cysteine before step b).

11. The method according to claim 1 wherein the first lectin and the second lectin is selected from Aleuria autantia (AAL), Galanthus nivalis (GNA), Hippeastrum hybrid (HHA), Narcissus pseudonarcissus (NPA), Sambucus nigra I (SNA-I), Ulex europaeus I (UEA-I), Urtica dioica (UDA), Allium savitum (ASA), Pisum sativum lectin (PSA), Phytohemagglutinin-L (PHA-L), and Pisum sativum lectin (PSA).

12. The method according to claim 1 wherein the first lectin and the second lectin is UDA.

13. The method according to claim 1 wherein the porous matrix comprises nitrocellulose.

14. The method according to claim 1 wherein the signal generation means is adapted to produce visually detectable color upon the binding.

15. The method according to claim 1 wherein the signal generation means comprises colloidal gold.

16. A method for determining traumatic brain injury (TBI) in a subject, the method comprising
a) providing
i. a probe comprising a porous matrix, the porous matrix comprising
a first part, a second part and a detection zone therebetween and
a first lectin adapted to bind a lectin-binding glycan indicative to TBI, and being immobilized to the detection zone, and
ii. a conjugate comprising
a second lectin adapted to bind the lectin-binding glycan indicative to TBI and
a signal generation means adapted to produce a detectable signal upon binding of the second lectin to the lectin-binding glycan immobilized to the detection zone via the first lectin,
wherein the first lectin and the second lectin have similar glycan-binding properties,
b) exposing the detection zone to a body fluid sample obtained from the subject,
c) exposing the detection zone to the conjugate,
d) detecting signal derived from the signal generation means on the detection zone,
e) determining level of the lectin-binding glycan in the body fluid sample based on the detecting,
f) comparing the level of the lectin-binding glycan in the body fluid sample to a control level indicative to a level of the lectin-binding glycan in body fluid of a subject not suffering from TBI and
g) determining TBI in the subject based on the comparing.

17. The method according to claim 16 comprising removing unbound material from the probe before the detecting of step d).

18. The method according to claim 16 wherein the body fluid is selected from saliva, urine, serum and blood plasma, tears, cerebrospinal fluid, oral secretions, respiratory secretions.

19. The method according to claim 16 wherein the body fluid is saliva, and the method comprises exposing the fluid sample to N-acetyl-L-cysteine before step b).

20. The method according to claim 16 wherein the first lectin and the second lectin is selected Aleuria autantia (AAL), Galanthus nivalis (GNA), Hippeastrum hybrid (HHA), Narcissus pseudonarcissus (NPA), Sambucus nigra I (SNA-I), Ulex europaeus I (UEA-I), Urtica dioica (UDA), Allium savitum (ASA), Pisum sativum lectin (PSA) and Phytohemagglutinin-L (PHA-L). According to another embodiment the first lectin and the second lectin is selected from Urtica dioica (UDA), Allium savitum (ASA), Pisum sativum lectin (PSA) and Phytohemagglutinin-L (PHA-L). According to a preferable embodiment the first lectin and the second lectin is UDA.

21. The method according to claim 16 wherein the first lectin and the second lectin is UDA.

22. The method according to claim 16 comprising
h) performing at least one neuroimaging procedure selected from a group consisting of x-ray, computerized tomography, and magnetic resonance imaging on the subject and determining the presence of TBI in the subject based on the elevated level of the lectin-binding glycan and the neuroimaging.
